(19) United States
(12) Patent Application Publication
(10) Pub. No.: US 2003/0134406 A1
(43) Pub. Date: Jul. 17, 2003

(54) Cellulose-active microorganisms

(76) Inventors: Andre Cesar Baeck, Bonheiden (BE); Alfred Busch, Londerzeel (BE); Michael Stanford Showell, Cincinnati, OH (US); Veerle de Boever, Lokeren (BE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/329,880

(22) Filed: Dec. 26, 2002

Related U.S. Application Data
(60) Provisional application No. 60/345,054, filed on Jan. 4, 2002.

(51) Int. Cl. C12N 1/20; C12Q 1/04; C12N 9/42

(52) U.S. Cl. 435/252.1; 435/34; 435/209

(57) ABSTRACT

Microorganisms that exhibit activity on cellulose and/or cellulose-containing materials are provided. Methods of using such microorganisms and/or agents produced by such microorganisms and screening methods for identifying such microorganisms are also provided.

### CROSS REFERENCE TO RELATED APPLICATIONS

This application claims priority to Application Serial No. 60/345,054, entitled “Cellulose-Active Microorganisms,” filed as a provisional patent application in the United States Patent and Trademark Office on Jan. 4, 2002.

### FIELD OF THE INVENTION

The present invention relates to microorganisms that exhibit activity on cellulose and/or cellulose-containing materials, screening methods for identifying such microorganisms and compositions and methods employing such microorganisms. More particularly, the present invention relates to microorganisms that exhibit activity on cellulose.

### BACKGROUND OF THE INVENTION

Cellulose is an abundant component in many non-living things that consumers e.g. use, wear, consume. For example, paper is made up of cellulose, as well as, textile, pulp, wood, plants, grass, fruit, vegetables, and other food wastes.

In several important industries like e.g. laundry, textile, pulp, paper, wood, plant, fruit industry there is continuous need to optimize the product and/or the process.

Means for decreasing the amount of waste, especially cellulose-containing wastes, due to the burden placed on landfills and the like to manage all the cellulose-containing wastes, formulators have been unsuccessfully trying to identify effective, safe and convenient means to degrade cellulose-containing wastes.

Accordingly, there exists a need to identify means for changing and/or degrading cellulose-containing materials that is effective, safe and convenient.

### SUMMARY OF THE INVENTION

The present invention fulfills the need identified above by providing microorganisms that exhibit activity on cellulose-containing materials, cellulose-active microorganisms, screening methods for identifying such microorganisms, methods for using such microorganisms to degrade cellulose and compositions comprising such microorganisms.

In one aspect of the present invention, a method for screening microorganisms to identify microorganisms that exhibit an acceptable enzymatic activity on cellulose-containing substrates, wherein the method comprises:

a) providing one or more microorganisms; and

b) screening said one or more microorganisms using a Screening Protocol described below; and

c) optionally, identifying said one or more microorganisms that exhibit acceptable enzymatic activity according to the Screening Protocol, is provided.

In another aspect of the present invention, microorganisms that exhibit activity on cellulose-containing materials.

In another aspect of the present invention, cellulose-active (i.e, degrading) microorganisms are provided.

In yet another aspect of the present invention, methods for treating cellulose-containing materials with an effective amount of a microorganism and/or enzyme produced by a microorganism in accordance with the present invention such that the cellulose-containing materials are degraded are provided.

In still another aspect of the present invention, systems for treating cellulose-containing materials (substrates) such that the cellulose is degraded are provided.

In still yet another aspect of the present invention, compositions comprising microorganisms in accordance with the present invention are provided.

In even yet another aspect of the present invention, microorganisms that are capable of producing an agent (i.e., enzyme, variant, mutation, etc.) that exhibits activity on cellulose is provided.

Accordingly, the present invention provides a method for screening microorganisms to identify microorganisms that exhibit activity on cellulose, cellulose-active microorganisms, methods for treating cellulose-containing materials with such microorganisms, and compositions comprising such microorganisms.

All percentages, ratios and proportions herein are on a weight basis based on a neat product unless otherwise indicated. All documents cited herein are hereby incorporated by reference.

### DETAILED DESCRIPTION OF THE INVENTION

Definitions:

“System” as used herein means a complex unity formed of many often, but not always, diverse parts (i.e., materials, compositions, devices, appliances, procedures, methods, conditions, etc.) subject to a common plan or serving a common purpose.

Deposit of Biological Material

A. Isolate N12 Pseudallescheria boydii strain was deposited under the terms of the Budapest Treaty in the Belgian Coordinated Collections of Microorganisms (herein “BCCM”), Mycothéque de l'Université Catholique de Louvain (herein “MUCL”) in Brussels, Belgium, on Jun. 9, 2000, and has been assigned Accession No. MUCL 42873. All restrictions on the availability of this deposit have been removed. More specifically, the strain will be irrevocably and without restriction or condition released to the public upon the issuance of a patent. The deposited strain is provided merely as convenience to those of skill in the art and is not an admission that a deposit is required for enablement, such as that required under 35 U.S.C. §112.

B. Isolate Q12 Pseudallescheria boydii strain was deposited under the terms of the Budapest Treaty in the Belgian Coordinated Collections of Microorganisms (herein “BCCM”), Mycothéque de l'Université Catholique de Louvain (herein “MUCL”) in Brussels, Belgium, on Jun. 9, 2000, and has been assigned Accession No. MUCL 42874. All restrictions on the availability of this deposit have been removed. More specifically, the strain will be irrevocably and without restriction or condition released to the public upon the issuance of a patent. The deposited strain is provided merely as convenience to those of skill in the art and is not an admission that a deposit is required for enablement, such as that required under 35 U.S.C. §112.

C. Isolate Z9 Sepedonium cfr. Chrysospermum strain was deposited under the terms of the Budapest Treaty in the Belgian Coordinated Collections of Microorganisms (herein “BCCM”), Mycothéque de l'Université Catholique de Louvain (herein “MUCL”) in Brussels, Belgium, on Jun. 9, 2000, and has been assigned Accession No. MUCL 42875. All restrictions on the availability of this deposit have been removed. More specifically, the strain will be irrevocably and without restriction or condition released to the public upon the issuance of a patent. The deposited strain is provided merely as convenience to those of skill in the art and is not an admission that a deposit is required for enablement, such as that required under 35 U.S.C. §112.

A license may be required to make, use or sell the deposited strains, and compounds derived therefrom, and no such license is hereby granted.

The deposits described above were made in accordance with the terms and provisions of the Budapest Treaty relating to deposit of microorganisms and were made for a term of at least thirty (30) years and at least five (5) years after the most recent request for the furnishing of a sample of the deposit is received by the depository, or for the effective term of the patent, whichever is longer, and will be replaced if it becomes non-viable during that period.

The microorganisms of the present invention may comprise fungal strains and/or mutants of fungal strains belonging to genera and/or species.

Screening Protocol:

The objective of the Screening Protocol is to identify new microorganisms which show high activity on cellulose-containing materials. In accordance with the present invention, the following two screening protocols are used for identifying such microorganisms. Microorganisms that satisfy at least one of the following screening protocols are within the scope of the present invention. Highly desirous microorganisms in accordance with the presnt invention satisfy both of the screening protocols. Especially suitable microorganisms are obtained from the genus Sepedonium and/or from the genus Pseudallescheria.

Protocol I—Cotton Fabric Screening Protocol

Step 1. Solid screening medium SSM: Prepare the following screening medium.

| Ingredient | | |
|---|---|---|
| NH4Cl | 5.0 | g/L |
| K2HPO4 | 5.0 | g/L |
| MgSO4 | 0.5 | g/L |
| CaCl2.2H2O | 0.5 | g/L |
| CuSO4.2H2O | 8 × 10⁻⁵ | g/L |
| FeSO4.7H2O | 0.02 | g/L |
| MnSO4.6H2O | 0.01 | g/L |
| ZnSO4.7H2O | 4 × 10⁻⁴ | g/L |
| Water | 1 | L |
| Agar | 20.0 | g/L |
| pH | 7.5 | |

Step 2. Screening experiment: Prepare small cotton muslin swatches [e.g. ˜5 cm×5 cm] prewashed with detergent [3×60° C. wash in Miele washing machine using Ariel Ultra powder detergent]. Sterilize swatches (Conditions for sterilization can be found in most handbooks of microbiology [see e.g. Dictionary of Microbiology and Molecular Biology—page 69—John Wiley & Sons—ISBN 0 471 94052 6]. A suitable method is autoclaving at 121° C. during 21 minutes at ˜1.1 atmosphere [1 atm=101.325 kPa]).

Prepare sterilized SSM [21’ at 121° C.]

Fill petri-dishes with the above screening medium [hot] and allow to solidify. When solidification starts, add one swatch per petri-dish.

Inoculate with purified strain.

Incubate at 30° C. during 10 days.

Recover swatch from petri-dish [e.g. using pair of tweezers], manually remove most of the agar from the swatch and wash the swatch in a Miele washing machine at 60° C. [water only].

Evaluate the fabric for visual damage and/or weight and/or tensile strength loss. Strains which show substantial effects on the cotton fabric are considered as suitable. By “substantial” effect we mean that the fabric either can not be recovered from the solid medium without visible damage [e.g. torn swatch, holes] and/or that the recovered fabric demonstrates high losses in weight [at least 10%, preferably at least 25%] and/or tensile strength loss [at least 10%, preferably at least 25%]. Tensile strength loss can be measured with any suitable method/instrument. For instance INSTRON equipment is very suitable to measure tensile strength loss. Krefeld cotton test fabric 11A is preferred but also cotton muslin is suitable.

Protocol II—Paper Protocol

Step 1. Liquid screening medium CM: Prepare the following liquid screening medium.

| Ingredient | | |
|---|---|---|
| Cellulose | 20.0 | g/L |
| [paper/see below] | | |
| (NH4)2SO4 | 2.5 | g/L |
| K2HPO4 | 1.0 | g/L |
| MgSO4 | 0.5 | g/L |
| Yeast extract | 1.0 | g/L |
| CuSO4.2H2O | 8 × 10⁻⁵ | g/L |
| FeSO4.7H2O | 0.02 | g/L |
| MnSO4.6H2O | 0.01 | g/L |
| ZnSO4.7H2O | 4 × 10⁻⁴ | g/L |
| Water | 1 | L |
| pH | 6.0 | |

Step 2. Screening experiment: Non-printed paper [newspaper quality] is shredded, sieved [to yield particles passing 2.3 mm<Ø<4.5 mm] and sterilized. Conditions for sterilization can be found in most handbooks of microbiology [see e.g. Dictionary of Microbiology and Molecular Biology—page 69—John Wiley & Sons—ISBN 0 471 94052 6]. A suitable method is autoclaving at 121° C. during 21 minutes at ˜1.1 atmosphere [1 atm=101.325 kPa].

20 g of such paper are added to one liter of sterilized liquid screening medium.

250 ml are added to a 500 ml erlenmeyer.

All but one erlenmeyers are inoculated with a purified strain. The non-inoculated erlenmeyer is used as a reference.

Subsequently, all the erlenmeyers are incubated at 30° C. and 200 RPM.

On a daily basis, the erlenmeyers are visually evaluated for paper degradation, in comparison to the reference erlenmeyer [not inoculated]. Strains which show substantial effects on the paper are considered as suitable. By “substantial” effect we mean that within 5 to 10 days and in comparison to the reference erlenmeyer, most of the paper disappeared [no longer perceivable when visually inspected].

Microorganisms:

Microorganisms identified by the Screening Protocol set forth above are within the scope of the present invention. Examples of such microorganisms are:

1) Genus Pseudallescheria:

Class: Ascomycota

Order: Microascales

Family: Microascaceae

Genus: Pseudalescheria

Other names for this genus are: Allescheria, Petriellidium, Monosporium [synonyms], Scedosporium, Graphium [anamorphic forms].

Nonlimiting examples of Pseudallescheria species: Ps. boydii, Ps. africana, Ps. angusta, Ps. desertarum, Ps. ellipsoidea, Ps. fimeti, Ps. Fusoidea.

Note that species also have synonym names [e.g. Ps. Boydii and Allescheria boydii, Petriellidium boydii] and occur in anamorph state [e.g. Ps. Boydii and anamorphs Graphium penicillioides, Graphium eumorphium, Monosporium apiospermum, Glenospora graphii, Scedosporium apiospermum, Stilbum basitruncatum, Sporocybe chartoikoon].

2) Genus Sepedonium:

Other names: Chaetomium, Hypomyces

Nonlimiting examples of Sepedonium species: Chaetomium piluliferum, Hypomyces chrysospermus, Sep. ampullosporum.

Note that species also have synonym names [Sep. albo-griseum, Sep. xylogenum, Sep. chrysospermum] and occur in anamorph state [e.g. Botryotrichum piluliferum].

Mutations

A mutation may occur spontaneously (i.e. spontaneous mutation) and/or may result from the activity of a mutagen (i.e., induced mutation). Some different types of nonlimiting mutations are forward or back mutation, insertion or deletion mutation, leaky mutation, mis-sense, nonsense or same-sense mutation, point, random or multisite mutation, polar mutation, suppressor mutation, etc. [see e.g. Dictionary of Microbiology and Molecular Biology—John Wiley & Sons—ISBN 0 471 94052 6].

Mutations can be induced in different ways. Chemical mutagens can be applied to generate mutants. Some examples are nitrous acid, hydroxylamine, methyl methane sulfonate, 2-aminopurine, nitrosoguanidine, etc.

Physical means can be used to obtain mutants, e.g. ionizing radiation (X-, beta-, gamma-rays), UV light, heat, etc.

Other mutagens induce frame shift mutagenesis, e.g. ICR compounds, proflavine, acridines, lead to the formation of mutants.

Obviously any other method known in the art can be applied to create mutants of the selected strains.

Mutants of the wildtype microorganisms are preferred when one/more of their properties is improved over the wildtype. For instance, improved enzymatic activity as a consequence of either increase specific activity and/or expression yield. Also other properties like e.g. pH optimum, stability, etc can be attractive challenges for mutation work. Target property changes are depending on the application condition.

Enzymes and variants

Enzymes are producible by the selected microorganism but can as well be cloned in host organisms, e.g. to improve expression, purity, etc.

Enzymes can be used in liquid preparation as well as in solid compounds. Nonlimiting examples of physical forms of composition in which the enzymes may be used are prills, granulates, agglomerates, pastes, gel, liquids, foams, powders, and tablets.

Obviously, enzymes can be modified by using the state of the art methods known to those of ordinary skill in the art, such as protein & genetic engineering and/or directed evolution.

Target for such modification is an improvement of the properties, i.e. adapting the enzyme to the conditions of the application so that it can perform better versus the wildtype enzyme. Some nonlimiting examples include:

higher specific activity

changed pH optimum

increase stability [versus e.g. temperature, composition ingredients, application environment]

oxidation stability

changed isoelectric point

Methods of Use

A microorganism and/or enzyme produced by a microorganism in accordance with the present invention may be used to degrade cellulose, especially cellulose-containing materials.

This includes industrial applications in areas such as textile, paper, pulp, fruit, vegetables, laundry and cleaning, declogging [drains, pipes, septic tanks, etc.], waste treatment, composting, etc.

Compositions

A microorganism and/or enzymes produced by a microorganism in accordance with the present invention may be incorporated into a composition. Such compositions can be in liquid, solid [e.g. granulated, stick, tablet, bar, powder, etc,] gel, paste, foam, etc. Liquid compositions can be aqueous or non-aqueous. The compositions may be concentrated or non-concentrated.

Microorganism can be used in any state know in the art, e.g active, dormant, lyophilized, etc.

Compositions in accordance with the present invention may further comprise nutrients, solvents, thickeners, surfactants, perfumes, dyes, clays, zeolites, enzyme stabilizers and other ingredients know in the art to transfer and/or carry microorganisms and/or enzymes onto application areas.

While particular embodiments of the subject invention have been described, it will be obvious to those skilled in the art that various changes and modifications of the subject invention can be made without departing from the spirit and scope of the invention. It is intended to cover, in the appended claims, all such modifications that are within the scope of the invention.

Having described the invention in detail with reference to nonlimiting embodiments, it will be clear to those skilled in the art that various changes and modifications may be made without departing from the scope of the invention and the invention is not to be considered limited to what is described in the specification.

What is claimed is:

1. A Pseudallescheria microorganism that exhibits activity on cellulose.

2. The microorganism according to claim 1 which comprises Pseudallescheria boydii.

3. The microorganism according to claim 2 wherein the Pseudallescheria boydii is selected from the group consisting of: isolate N12 Pseudallescheria boydii strain (MUCL 42873), isolate Q12 Pseudallescheria boydii strain (MUCL 42874) and mixtures thereof.

4. The microorganism according to claim 1 wherein the microorganism is capable of producing an agent that exhibits activity on cellulose.

5. A cellulose-active composition comprising a microorganism according to claim 1 and/or agents produced by such microorganism.

6. A Sepedonium cfr. Chrysospermum microorganism that exhibits activity on cellulose.

7. The microorganism according to claim 6 which comprises isolate Z9 Sepedonium cfr. Chrysospermum strain (MUCL 42875).

8. The microorganism according to claim 6 wherein the microorganism is capable of producing an agent that exhibits activity on cellulose.

9. A cellulose-active composition comprising a microorganism according to claim 6 and/or agents produced by such microorganism.

10. A method for screening microorganisms to identify microorganisms that exhibit an acceptable enzymatic activity on cellulose-containing substrates, the method comprises:
a) providing one or more microorganisms; and
b) screening said one or more microorganisms using a Screening Protocol described herein; and
c) optionally, identifying said one or more microorganisms that exhibit acceptable enzymatic activity according to the Cotton Fabric Test.

11. The method according to claim 10 wherein said one or more microorganisms comprise a fungal strain.

12. The method according to claim 11 wherein said one or more microorganisms comprise a fungal strain belonging to the genus Sepedonium and/or Pseudallescheria.

13. A method for degrading cellulose-containing substrates into hydrolysates comprising contacting said cellulose with a fungal strain belonging to the genus Sepedonium and/or the genus Pseudallescheria.

14. An enzyme that exhibits an acceptable enzymatic activity on cellulose-containing substrates and is produced by a microorganism belonging to a fungal strain belonging to the genus Sepedonium and/or the genus Pseudallescheria.

15. An enzyme according to claim 14 wherein said enzyme is cloned in a host organism.

16. A mutant of an enzyme according to claim 14.

17. A mutant enzyme according to claim 16 wherein said mutant enzyme is cloned in a host organism.
